(19) United States
(12) Patent Application Publication
(10) Pub. No.: US 2003/0147817 A1
(43) Pub. Date: Aug. 7, 2003

(54) Blended toothpaste formulas and method for manufacture

(76) Inventor: Derrik Deinas, Bremen (DE)

Correspondence Address: FELIX L. FISCHER, ATTORNEY AT LAW, 1607 MISSION DRIVE, SUITE 204, SOLVANG, CA, 93463, US

(21) Appl. No.: 10/316,140

(22) Filed: Dec. 9, 2002

(30) Foreign Application Priority Data
Dec. 10, 2001 (DE) DE 201 19 966 U1

(51) Int. Cl. A61K 7/16; A61K 7/26

(52) U.S. Cl. 424/49; 424/58

(57) ABSTRACT

A toothpaste having a stimulant added to the formulation for absorption through the gums and membranes in the mouth during brushing for oral hygiene.

### CROSS-REFERENCE TO RELATED APPLICATIONS

This application claims priority under 35 U.S.C. 119 of German Utility Patent (Gebrauchsmusterschrift) DE 201 19 966 U1 entitled MIT BELEBENDEN STOFFEN ANGEREICHERTE ZAHNPASTA filed on Dec. 10, 2001.

### BACKGROUND OF THE INVENTION

1. Field of the Invention

This invention relates generally to the field of dental pastes and, more particularly, to toothpaste with a stimulant blended into the paste.

2. Description of the Related Art

Many people enjoy the invigorating effects of caffeine or other natural stimulants in beverages such as coffee or tea. Particularly in the morning, such beverages, taken with breakfast or alone, help awaken the person and assist in preparing for the day ahead. However, on some occasions, the press of time prevents preparation and consumption of such beverages, particularly for individuals who may skip the breakfast meal. Other individuals may not like coffee or tea. In some cases, stimulants may be taken as pills or chewable lozenges to combat drowsiness, however, certain individuals may find these forms too strong or do not wish to keep such forms of stimulant available for use.

Toothpastes have previously been employed to provide various beneficial chemical treatments for users such as fluoride and whitening formulations.

It is therefore desirable to make a mild stimulant available for use in a form which is easily adapted to the lifestyle of each individual.

### SUMMARY OF THE INVENTION

The present invention allows early morning hygiene in the form of brushing one's teeth to also provide a mild stimulant for assistance in alleviating morning drowsiness. A toothpaste with stimulant blended into the formulation allows the stimulant to be absorbed through the gums and membranes in the mouth and, in minor quantities, swallowed during brushing and rinsing of the teeth. In alternative embodiments caffeine or coffee extract, teein or tea-extract, Taurin or Guarana are employed as the stimulant. In certain embodiments, vitamin C is also added for additional therapeutic benefit to the user.

### DETAILED DESCRIPTION OF THE INVENTION

The invention employs a standard toothpaste mixture into which a stimulant, in a first embodiment caffeine, is added. The caffeine is mixed directly into the paste during formulation in certain embodiments. In a second embodiment, the stimulant, in the form of concentrated coffee or coffee extract, is entrained in the toothpaste formulation during extrusion into the tube in a manner similar to color gel entrainment in children's toothpastes. Entrainment allows complete preparation of the toothpaste formula which can then be used either with the added stimulant or without to produce toothpaste with our without stimulant but having similar chemical constituents. Consumers may purchase the same toothpaste with and without stimulant for use in the morning and evening respectively.

In alternative embodiments, the stimulant employed is concentrated tea extract or teein, Taurin or Guarana. Mixture or entrainment of the stimulant is accomplished as previously defined for the caffeine/coffee embodiments. The flavor of the toothpaste may be nominally affected by the stimulant selected and multiple formulations are employed to allow selection of flavor by the consumer. Additionally, certain stimulants may have differing effects on various individuals. Therefore, offering multiple formulations allows selection of the particular blended toothpaste most efficacious for the user.

For added benefit, Vitamin C is added to the toothpaste formulation to avert potential vitamin C depletion or deficiency which may be associated with stimulants such as caffeine. As with the stimulant, the Vitamin C in powdered or liquid form is mixed in the paste during formulation or entrained during extrusion into the tube.

In use, the invention is applied to a toothbrush in the normal fashion and the teeth and gums are brushed as normally recommended for oral hygiene and cavity prevention. Since the stimulant ingredients are rapidly absorbed through the gums and mucous membranes in the mouth and tongue, brushing in this manner is sufficient to provide a pleasant stimulating effect.

Having now described the invention in detail as required by the patent statutes, those skilled in the art will recognize modifications and substitutions to the specific embodiments disclosed herein. Such modifications are within the scope and intent of the present invention as defined in the following claims.

What is claimed is:

1. A blended toothpaste comprising:
a standard toothpaste base; and
a stimulant added to the toothpaste base.

2. A toothpaste as defined in claim 1 wherein the stimulant is caffeine.

3. A toothpaste as defined in claim 1 wherein the stimulant is coffee extract.

4. A toothpaste as defined in claim 1 wherein the stimulant is teein.

5. A toothpaste as defined in claim 1 wherein the stimulant is tea extract.

6. A toothpaste as defined in claim 1 wherein the stimulant is Taurin.

7. A toothpaste as defined in claim 1 wherein the stimulant is Guarana.

8. A toothpaste as defined in claim 1 further comprising Vitamin C added to the toothpaste base.

9. A method for formulating a blended toothpaste comprising the steps of:
mixing a standard toothpaste base;
blending into the standard toothpaste base a stimulant.

10. A method for formulating a blended toothpaste as defined in claim 9 wherein the step of blending further comprises the step of selecting a stimulant from the group consisting of caffeine, coffee extract, teein, tea extract, Taurin and Guarana.

11. A method for formulating a blended toothpaste as defined in claim 9 wherein the step of blending comprises entraining the stimulant in a concentrated form into the toothpaste base during extrusion into a packaging tube.
